(19) United States
(12) Patent Application Publication
(10) Pub. No.: US 2017/0073283 A1
(43) Pub. Date: Mar. 16, 2017

(54) ENHANCED PERFORMANCE OF THE DEHYDROGENATION BY THE REDUCTION OF COKE FORMATION USING PRE-ACTIVATED CO2

(71) Applicant: SABIC GLOBAL TECHNOLOGIES B.V., Bergen op Zoom (NL)

(72) Inventors: YongMan Choi, Riyadh (SA); Adel Abdullah Al-Ghamdi, Riyadh (SA); Ramsey Bunama, Riyadh (SA); Khalid M. El-Yahyaoui, Riyadh (SA)

(73) Assignee: Sabic Global Technologies B.V., Bergen op Zoom (NL)

(21) Appl. No.: 15/308,880

(22) PCT Filed: May 5, 2015
(86) PCT No.: PCT/IB2015/053285

§ 371 Date: Nov. 4, 2016

Related U.S. Application Data
(60) Provisional application No. 61/989,127, filed on May 6, 2014.

(51) Int. Cl. C07C 5/333; H05H 1/24; B01J 19/08

(52) U.S. Cl. CPC C07C 5/333; B01J 19/088; H05H 1/2406; B01J 2219/0805; B01J 2219/0894; B01J 2219/0871; B01J 2219/00123

(57) ABSTRACT

The present disclosure addresses the deficiencies described above by providing systems and methods for enhancing the efficiency and yield of alkene production. The methods and systems provide for the use of activated CO₂ in a dehydrogenation reactor along with an alkane stream. Through the use of the methods and systems of the invention, catalyst deactivation by coke deposition is reduced and the selectivity and efficiency of the dehydrogenation reaction is improved.

### TECHNICAL FIELD

The present disclosure relates generally to methods for enhancing the performance of a dehydrogenation reactor, in particular by reducing catalyst deactivation due to coke deposition.

### BACKGROUND

Alkane dehydrogenation is a recognized process for production of a variety of useful hydrocarbon products, such as isobutylene for conversion to MTBE and propylene for use in the polymer industry. There are several current catalytic processes useful for catalytic dehydrogenation of light alkanes, including the Sud-Chemie CATOFIN® process, UOP's Oleflex® process, Phillips' Star™ process and the Snamprogetti-Yarsintez process.

The dehydrogenation of alkanes proceeds via a reversible chemical reaction involving the breaking of two hydrocarbon-hydrogen bonds with the concomitant formation of a hydrogen molecule and a molecule containing a double carbon bond. Despite the apparent simplicity of this reaction, it is one of the most complex chemical processes to achieve industrially. Because dehydrogenation reactions are highly endothermic, they require the addition of a large amount of heat to obtain acceptable yields. However, these high temperatures enhance undesired parallel side reactions, including the formation of coke on the catalyst bed.

The formation of coke on catalysts resulting from decomposition of hydrocarbon feeds is a widely studied issue in the petrochemical industry because catalyst deactivation by coke build-up adversely affects the catalyst performance, leading to lower yields and expensive maintenance. For example, once catalysts are deactivated, they have to be taken off line and regenerated by means of extra reactions, such as an oxidation reaction like below:

C(a)+O₂(g)→CO₂(g) (1)

where the subscripts of “a” and “g” are an adsorbed species on catalyst surfaces and gas phase, respectively. However, regeneration of catalyst using this reaction is both costly and time-consuming. It would be desirable to have a way of running dehydrogenation reactions under conditions that would not require such high temperatures and that would minimize the formation of coke on the catalyst.

### SUMMARY OF THE INVENTION

Disclosed, in various embodiments, are systems and methods for obtaining an alkylene.

A method for obtaining an alkene, comprising: admitting into a dehydrogenation reactor, via a first inlet, a first reactant stream comprising an alkane; admitting into the dehydrogenation reactor, via a second inlet, a second reactant stream comprising activated CO₂, reacting the first reactant stream and second reactant stream over a dehydrogenation catalyst in the dehydrogenation reactor under conditions to convert the alkane into an alkene; and recovering the alkene.

A system for producing an alkene by dehydrogenating an alkane, comprising: a dehydrogenation reactor with a dehydrogenation catalyst contained therein, said dehydrogenation reactor being configured to run under conditions to promote dehydrogenation of an alkane, wherein the dehydrogenation reactor comprises a first inlet configured to receive an alkane stream from a first source; and a second inlet configured to receive activated CO₂ from a second source; an outlet configured to permit recovery of the alkene.

These and other features and characteristics are more particularly described below.

### BRIEF DESCRIPTION OF THE DRAWINGS

The following is a brief description of the drawings wherein like elements are numbered alike and which are presented for the purposes of illustrating the exemplary embodiments disclosed herein and not for the purposes of limiting the same.

FIG. 1 is a flowchart depicting a method for obtaining an alkene using a plasma reactor to provide activated CO₂ to the dehydrogenation reactor in accordance with an exemplary implementation of the present disclosure.

### DETAILED DESCRIPTION OF THE INVENTION

This invention provides novel methods and systems for obtaining an alkene using activated CO₂ which provides for more selective and efficient dehydrogenation with reduced coke deposition than conventional dehydrogenation methods.

The present disclosure addresses the deficiencies described above by providing systems and methods for enhancing the efficiency and yield of alkene production. In one aspect, the methods and systems provide for the use of plasma-activated CO₂ in a dehydrogenation reactor along with an alkane stream. Through the use of the methods and systems of the invention, catalyst deactivation by coke deposition is reduced and the selectivity and efficiency of the dehydrogenation reaction is improved. The methods and systems of the invention can be advantageously used to convert alkanes to more commercially valuable alkenes. One example of such a conversion is the conversion of isobutane to isobutene. The isobutene produced in this manner can be used as a reactant stream to produce other valuable petrochemical products, such as methyl tertiary butyl ether (MTBE), which is commonly added to gasoline as an anti-knocking additive.

In one aspect, the invention provides a method for obtaining an alkene comprising admitting into a dehydrogenation reactor a first reactant stream comprising an alkane; admitting into the dehydrogenation reactor a second reactant stream comprising activated CO₂; reacting the first reactant stream and second reactant stream over a dehydrogenation catalyst in the dehydrogenation reactor under conditions to convert the alkane into an alkene; and recovering the alkene. Preferably, the activated CO₂ is generated by a plasma reactor. If desired, the dehydrogenation catalyst is mixed with a heat-generating material that increases the efficiency of the dehydrogenation reaction.

In yet another aspect, the invention provides system for obtaining an alkene, the system comprising a dehydrogenation reactor with a dehydrogenation catalyst contained therein, configured to run under conditions to promote dehydrogenation of an alkane, wherein the dehydrogenation reactor comprises, a first inlet configured to receive an alkane stream from a first source, a second inlet configured to receive activated CO₂ from a second source; and an outlet configured to permit recovery of the alkene. In an embodiment, the system includes a plasma reactor for providing activated CO₂. In a particular embodiment, the plasma reactor is configured to provide activated CO₂ to the dehydrogenation reactor via the second inlet.

Generally, the activated CO₂ of the invention can be produced by any source that is capable of creating plasma. In certain preferred embodiments, the activated CO₂ is produced by a non-thermal plasma reactor, as described herein. As the skilled artisan will appreciate, the chemical reactions that occur within a plasma reactor are quite complicated, involving molecules, atoms, ions, radicals, and/or electrons. For instance, one exemplary reaction that produces activated CO₂ is as follows:

CO₂+e→CO₂*+e (2)

In Reaction (2), the electron (“e”) on the reactant side is highly energetic and produced by the plasma reactor. The plasma activated CO₂ is denoted as “CO₂*” on the product side of Reaction (2).

The activated CO₂ species produced by a plasma reactor is itself highly reactive and can even undergo decomposition reactions. For example, one such decomposition reaction involves the reaction of an energetic electron with CO₂ to produce carbon monoxide (CO) and atomic oxygen (O) decomposition products according to the following reaction:

CO₂+e→CO+O+e (3)

The decomposition products formed by this reaction can, in turn, be consumed in subsequent reactions. An example of such a reaction includes the following;

O₍ₚ₎+H₂→H₂O (4),

where O₍ₚ₎ refers to atomic oxygen produced by the plasma reactor and the hydrogen (H₂) is formed during dehydrogenation reactions, as described herein.

One aspect of the invention is the recognition that activated CO₂ (CO₂*) formed in a plasma reactor can be a useful for limiting coke formation on catalysts during dehydrogenation. Without wishing to be limited by theory, it is believed that the activated CO₂ is capable of reacting with intermediate chemical species formed during the dehydrogenation reaction, thereby suppressing undesirable side reactions that lead to coke formation. For example, the dehydrogenation of isobutane (i-C₄H₁₀) to form isobutene (i-C4H₈) also produces by-product species, including propane (C₃H₈), propene (C₃H₆), ethane (C₂₆), ethylene (C₂H₄), methane (CH₄), hydrogen (H₂), and coke. Elementary reactions that lead to the formation of these species include the following:

i-C₄H₁₀→i-C₄H₈+H₂ (5)

i-C₄H₁₀+H₂→C₃H₈+CH₄ (6)

C₃H₈→C₃H₆+H₂ (7)

2CH₄→C₂H₆+H₂ (8)

C₃H₈→C₂H₄+CH₄ (9)

C₂H₆→C₂H₄+H₂ (10)

coke formation. (11)

The invention recognizes that the formation of propane (C₃H₈) and other decomposition products from isobutane (i-C₄H₁₀) requires the presence of hydrogen (H₂) (see, e.g., Reaction (6)). Thus, the invention recognizes that it is advantageous to provide alternate reaction pathways for hydrogen, so that decomposition reactions (e.g., reactions (6)-(11)) are suppressed in accordance with Le Chatelier's principle. For example, the hydrogen can be consumed via the reverse-water-gas-shift (RWGS) reaction

CO₂+H₂→CO+H₂O. (12)

The RWGS reaction, however, is reversible and has a relatively high barrier to reaction, owing to the stability of carbon dioxide. Accordingly, the present invention contemplates using activated CO₂ as a reactant in the RWGS reaction,

CO₂*+H₂→CO+H₂O, (13)

which drives the reaction towards the formation of carbon monoxide and water. In addition, the carbon monoxide that is produced by the RWGS reaction can be used to generate heat via a reaction with a heat-generating material. For example, the heat-generating material can comprise copper (II) oxide, which is reduced by CO to produce copper metal according to the following exothermic reaction.

CO+CuO→CO₂+Cu, ΔH=−126.9 kJ/mol (14)

The heat generated by the reaction of the heat-generating material with CO results in more efficient conversion of the alkane (e.g., isobutane) to alkene (e.g., isobutene) in the dehydrogenation reactor. If desired, the CO₂ produced via the reduction of copper (II) oxide can be separated to produce a CO₂ stream that serves as an input stream for the plasma reactor.

In certain preferred implementations of the invention, the activated CO₂ is produced by a plasma reactor. The type of plasma reactor is not particularly limited and typically is selected based on operational parameters of the particular dehydrogenation system in question. Non-limiting examples of such parameters include the physical configuration of the system, the desired operating pressures, and/or the desired flux of activated CO₂. In certain exemplary implementations, the plasma reactor generates a non-thermal plasma. One example of such a plasma reactor is a dielectric barrier discharge (DBD) reactor. Such reactors are known in the art. See, e.g., Liu et al. “Converting of Carbon Dioxide into More Valuable Chemical using Catalytic Plasmas,” Fuels, 45(4), 694-697, the contents of which are incorporated by reference in their entirety. One exemplary DBD plasma reactor configuration comprises two concentric tubes arranged such that the gas flows along the annular gap between the tubes. In certain implementations, the outer tube is made of metal (e.g., stainless steel) and the inner tube is made of a dielectric material (e.g., quartz). The lengths of these tubes are equal and can fall in the range of 50-300 millimeters (mm). The annular gap between the tubes typically is approximately 1 mm. The plasma is ignited between the annular gap between the two tubes using a high voltage generator operating at about 25 kiloHertz (kHz).

In addition to the dielectric barrier discharge reactor, the invention also contemplates the use of other types of non-thermal plasma reactors, including glow discharge, corona discharge, silent discharge, microwave discharge, and radio frequency discharge reactors. As the skilled artisan will appreciate, the configurations of some non-thermal plasma reactors limit them to low pressure operation, which can be appropriate only under certain reactor conditions. For example, a glow discharge plasma reactor typically operates at low pressure (about 10 millibar (mbar)), making it less preferred for high-pressure, high-throughput systems. By contrast, corona discharge plasma reactors and silent discharge plasma reactors typically operate at pressures of about 1 bar.

The alkane for use in accordance with the inventive methods is not particularly limited. In preferred implementations, the alkane is a C₂-C₁₀ alkane, and more preferably a C₃ to C₅ alkane. The alkane can be a straight chain alkane or a branched alkane. In one particularly preferred embodiment, the alkane is isobutane.

The dehydrogenation catalysts of the inventive methods and systems are not particularly limited and include any dehydrogenation catalysts known in the art. Non-limiting examples of catalysts suitable for use in the dehydrogenation processes contemplated by the invention include Group VIII metals (e.g., Pt/Sn on alumina, with promoters); chromium oxides supported on alumina or zirconium (preferably with promoters); supported iron oxides (with promoters); supported gallium catalysts (e.g., on mordenite, SAPO-11, MCM-41 or alumina). In particularly preferred embodiments, the catalyst used is a chromium oxide based catalyst, which preferably is supported as described above.

In certain implementations of the invention, the dehydrogenation catalyst is combined with a heat generating material, typically as a physical mixture. The invention recognizes that the presence of a heat generating material in the catalytic bed advantageously raises the local temperature and promotes the dehydrogenation reaction to form alkenes. The heat generating material is not particularly limited and preferred heat generating materials are those that are capable of reacting exothermically with chemical species present in the dehydrogenation reactor system without substantially interfering with the desired dehydrogenation reaction. For example, in certain embodiments, the heat generating materials are metal oxide materials that they can react with chemical species that are produced in the system used to run the dehydrogenation reaction. For instance, such metal oxide materials can include comprise copper (II) oxide (see, e.g., Reaction (14)). Other examples of heat-generating materials those recited in U.S. Pat. No. 8,188,328, which is hereby incorporated by reference in its entirety. Preferably, the heat-generating material is present in an amount sufficient to increase the efficiency of the dehydrogenation reaction throughout the catalyst bed. For example, in certain implementations of the invention, the concentration of heat-generating material present in the catalyst bed is 0.5 to 30 weight percent (wt. %), more preferably 1 to 25 wt. %, and even more preferably 5 to 15 wt. %. In one particularly preferred implementation, the dehydrogenation catalyst is a supported chromium oxide based catalyst, the dehydrogenation catalyst present in an amount of 85-95 wt. %, and the concentration of heat-generating material in the catalyst bed is 5-15 wt. %.

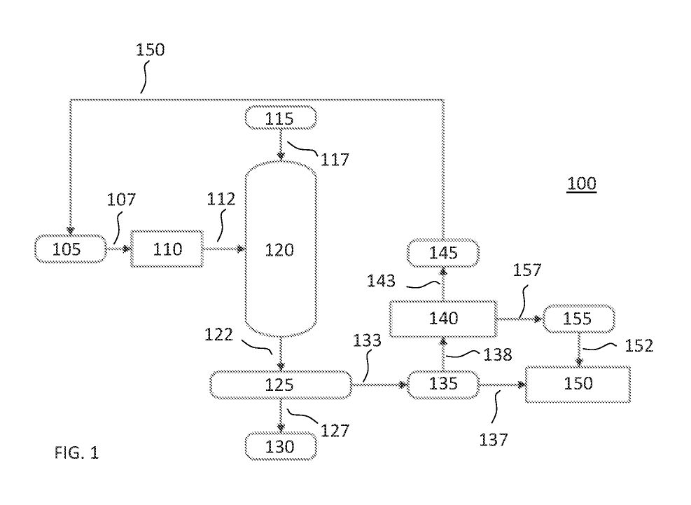

FIG. 1 shows a schematic diagram of a process and a system 100 according to one exemplary implementation of the invention. In FIG. 1, CO₂ source 105 supplies CO₂ inlet stream 107 to plasma reactor 110. An activated CO₂ stream 112 is produced by plasma reactor 110 and admitted into catalytic reactor chamber 120, which contains a catalyst which comprises a dehydrogenation catalyst and optionally a heat-generating material. Alkane source 115 provides reactant alkane stream 117 to catalytic reactor chamber 120, where it undergoes a dehydrogenation reaction over catalyst to form product stream 122. Product stream 122 contains the alkene formed by the dehydrogenation reaction, as well as chemical species that are produced, for example, by plasma reactor 110. In preferred embodiments, alkane source 115 provides isobutane in alkane stream 117 for conversion into isobutene. FIG. 1 shows that product stream 122 produced by catalytic reactor chamber 120 is separated into the alkene product stream 127 (e.g., an isobutene stream) leading to alkene product 130 (e.g., isobutene). By-product stream 133, which comprises CO, is fed to separation unit 135, which provides reactant stream 138 to water-gas-shift (WGS) reactor 140. Within WGS reactor 140, the CO from by-product stream 133 can be reacted with water to produce product stream 143, which comprises CO₂ product 145. If desired, CO₂ product 145 may be recycled to CO₂ source 105 via conduit 150. In addition (or in the alternative), the CO₂ produced by WGS reactor 140 shown as line 157 can be combined with hydrogen from hydrogen source 155 to form stream 152, which is then combined with CO stream 137 from separation unit 135 to produce syngas 150.

The methods and systems for obtaining an alkylene disclosure herein include at least the following embodiments:

Embodiment 1: A method for obtaining an alkene, comprising: admitting into a dehydrogenation reactor, via a first inlet, a first reactant stream comprising an alkane; admitting into the dehydrogenation reactor, via a second inlet, a second reactant stream comprising activated CO₂, reacting the first reactant stream and second reactant stream over a dehydrogenation catalyst in the dehydrogenation reactor under conditions to convert the alkane into an alkene; and recovering the alkene.

Embodiment 2: The method according to Embodiment 1, wherein the activated CO₂ is produced by a plasma reactor.

Embodiment 3: The method according to Embodiment 2, wherein the plasma reactor is a non-thermal plasma reactor selected from a dielectric barrier discharge reactor, a glow discharge reactor, a corona discharge reactor, a silent discharge reactor, a microwave discharge reactor, and a radio frequency discharge reactor.

Embodiment 4: The method according to Embodiment 3, wherein the plasma reactor is a dielectric barrier discharge reactor.

Embodiment 5: The method according to any of Embodiments 1-4, wherein the dehydrogenation catalyst is physically mixed with a heat-generating material.

Embodiment 6: The method according to Embodiment 5, wherein the heat-generating material comprises copper (II) oxide.

Embodiment 7: A system for producing an alkene by dehydrogenating an alkane, comprising: a dehydrogenation reactor with a dehydrogenation catalyst contained therein, said dehydrogenation reactor being configured to run under conditions to promote dehydrogenation of an alkane, wherein the dehydrogenation reactor comprises a first inlet configured to receive an alkane stream from a first source; and a second inlet configured to receive activated CO₂ from a second source; an outlet configured to permit recovery of the alkene.

Embodiment 8: The system according to Embodiment 7, wherein the second source is a plasma reactor.

Embodiment 9: The system according to Embodiment 8, wherein the plasma reactor is a non-thermal plasma reactor selected a dielectric barrier discharge reactor, a glow discharge reactor, a corona discharge reactor, a silent discharge reactor, a microwave discharge reactor, and a radio frequency discharge reactor.

Embodiment 10: The system according to any of Embodiments 7-9, wherein the dehydrogenation catalyst is physically mixed with a heat-generating material.

Embodiment 11: The system according to Embodiment 10, wherein the heat generating material is a metal oxide.

Embodiment 12: The system according to Embodiment 11, wherein the heat-generating material comprises copper (II) oxide.

Embodiment 13: The system according to any of Embodiments 7-12, wherein the dehydrogenation catalyst is a supported chromium oxide based catalyst.

Embodiment 14: The system according to any of Embodiments 7-13, wherein the dehydrogenation catalyst is present in an amount of 85-95 wt. %.

Embodiment 15: The system according to any of Embodiments 7-14, wherein the heat-generating material is present in a catalyst bed in the dehydrogenation reactor in an amount of 0.5 to 30 wt. %.

Embodiment 16: The system according to Embodiment 15, wherein the concentration of heat-generating material in the catalyst bed is 5 to 15 wt. %.

Embodiment 17: The system according to any of Embodiments 7-16, wherein the alkane is a C₂-C₁₀ alkane.

Embodiment 18: The system according to Embodiment 17, wherein the alkane is a C₃ to C₅ alkane.

Embodiment 19: The system according to Embodiment 17 or Embodiment 18, wherein the alkane is isobutane.

The exemplary methods, and systems described in this disclosure are illustrative and, in alternative implementations, certain steps can be performed in a different order, in parallel with one another, omitted entirely, and/or combined between different exemplary implementations, and/or certain additional acts can be performed, without departing from the scope and spirit of this disclosure. Accordingly, such alternative implementations are included in the inventions described herein.

Although specific implementations have been described above in detail, the description is merely for purposes of illustration. It should be appreciated, therefore, that many aspects described above are not intended as required or essential elements unless explicitly stated otherwise. Various modifications of, and equivalent acts corresponding to, the disclosed aspects of the exemplary implementations, in addition to those described above, can be made by a person of ordinary skill in the art, having the benefit of the present disclosure, without departing from the spirit and scope of the invention defined in the following claims, the scope of which is to be accorded the broadest interpretation so as to encompass such modifications and equivalent structures.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “±10%” means that the indicated measurement can be from an amount that is minus 10% to an amount that is plus 10% of the stated value. The terms “front”, “back”, “bottom”, and/or “top” are used herein, unless otherwise noted, merely for convenience of description, and are not limited to any one position or spatial orientation. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs. A “combination” is inclusive of blends, mixtures, alloys, reaction products, and the like.

All cited patents, patent applications, and other references are incorporated herein by reference in their entirety. However, if a term in the present application contradicts or conflicts with a term in the incorporated reference, the term from the present application takes precedence over the conflicting term from the incorporated reference

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

I/We claim:

1. A method for obtaining an alkene, comprising:
admitting into a dehydrogenation reactor, via a first inlet, a first reactant stream comprising an alkane;
admitting into the dehydrogenation reactor, via a second inlet, a second reactant stream comprising activated CO₂,
reacting the first reactant stream and second reactant stream over a dehydrogenation catalyst in the dehydrogenation reactor under conditions to convert the alkane into an alkene; and recovering the alkene.

2. The method according to claim 1, wherein the activated CO₂ is produced by a plasma reactor.

3. The method according to claim 2, wherein the plasma reactor is a non-thermal plasma reactor selected from a dielectric barrier discharge reactor, a glow discharge reactor, a corona discharge reactor, a silent discharge reactor, a microwave discharge reactor, and a radio frequency discharge reactor.

4. The method according to claim 3, wherein the plasma reactor is a dielectric barrier discharge reactor.

5. The method according to claim 1, wherein the dehydrogenation catalyst is physically mixed with a heat-generating material.

6. The method according to claim 5, wherein the heat-generating material comprises copper (II) oxide.

7. A system for producing an alkene by dehydrogenating an alkane, comprising:
a dehydrogenation reactor with a dehydrogenation catalyst contained therein, said dehydrogenation reactor being configured to run under conditions to promote dehydrogenation of an alkane, wherein the dehydrogenation reactor comprises
a first inlet configured to receive an alkane stream from a first source; and
a second inlet configured to receive activated CO₂ from a second source;
an outlet configured to permit recovery of the alkene.

8. The system according to claim 7, wherein the second source is a plasma reactor.

9. The system according to claim 8, wherein the plasma reactor is a non-thermal plasma reactor selected a dielectric barrier discharge reactor, a glow discharge reactor, a corona discharge reactor, a silent discharge reactor, a microwave discharge reactor, and a radio frequency discharge reactor.

10. The system according to claim 7, wherein the dehydrogenation catalyst is physically mixed with a heat-generating material.

11. The system according to claim 10, wherein the heat generating material is a metal oxide.

12. The system according to claim 11, wherein the heat-generating material comprises copper (II) oxide.

13. The system according to claim 7, wherein the dehydrogenation catalyst is a supported chromium oxide based catalyst.

14. The system according to claim 7, wherein the dehydrogenation catalyst is present in an amount of 85-95 wt. %.

15. The system according to claim 7, wherein the heat-generating material is present in a catalyst bed in the dehydrogenation reactor in an amount of 0.5 to 30 wt. %.

16. The system according to claim 15, wherein the concentration of heat-generating material in the catalyst bed is 5 to 15 wt. %.

17. The system according to claim 7, wherein the alkane is a C₂-C₁₀ alkane.

18. The system according to claim 17, wherein the alkane is a C₃ to C₅ alkane.

19. The system according to claim 17, wherein the alkane is isobutane.
